(19) United States
(12) Patent Application Publication
(10) Pub. No.: US 2013/0123732 A1
(43) Pub. Date: May 16, 2013

(54) LUMINESCABLE RELEASE OR PROTECTIVE FILM

(71) Applicant: HUHTAMAKI FILMS GERMANY GMBH & CO. KG, Forchheim (DE)

(72) Inventors: Stefan SITZMANN, Kirchehrenbach (DE); Georg DISTLER, Waischenfeld (DE)

(73) Assignee: HUHTAMAKI FILMS GERMANY GMBH & CO. KG, Forchheim (DE)

(21) Appl. No.: 13/721,239

(22) Filed: Dec. 20, 2012

Related U.S. Application Data
(63) Continuation of application No. PCT/EP2011/002802, filed on Jun. 8, 2011.

(30) Foreign Application Priority Data
Jun. 22, 2010 (DE) 10 2010 024 638.7

(51) Int. Cl. B32B 37/00; A61F 13/56; B32B 38/00

(52) U.S. Cl. CPC B32B 37/0076; B32B 38/00; A61F 13/5605
USPC 604/385.05; 428/29; 156/64; 156/378; 493/331

(57) ABSTRACT

A release film or protective film having a silicone coating composed at least at regular intervals of a luminescable silicone coating section, method for furnishing starting products with such a release or protective film, products produced by this method, and device for implementing the aforementioned method.

This application is a Continuation of PCT/EP2011/002802, filed Jun. 8, 2011, which claims foreign priority benefit under 35 U.S.C.§119 of German Patent Application 10 2010 024 638.7 filed Jun. 22, 2010, the contents of all of which are incorporated herein by reference.

The present invention relates to a release film or protective film whose silicone coating is respectively composed, at least at regular intervals, of a luminescent silicone coating section, to a process for equipping ingoing products with this type of release film or protective film, to products produced by said process, and moreover to an apparatus for carrying out the abovementioned process.

### BACKGROUND OF THE INVENTION

Release films are widely used as releasable protective films for adhesive tapes, self-adhesive labels or adhesive sanitary and hygiene items, in order to prevent adhesion of these single- or double-sided-adhesive products during storage or processing, and must feature good release action in relation to adhesives such as pressure-sensitive adhesives, or other adhesive materials.

Siliconized release films have already been disclosed in the prior art, for example in EP 1 277 802 A1 or EP 0 769 540 A2.

For the application of release films of this type equipped with an adhesive onto ingoing products, such as ingoing sanitary or hygiene products, or vice versa, there are usually colored registration marks, i.e. colored markings applied, for example, by printing at certain intervals on the siliconized area of the release film, which permit precise positioning of the release films and of the ingoing products in relation to one another during the production of products such as sanitary or hygiene products comprising this type of release film, in an apparatus intended for this purpose.

However, a disadvantage in the production of products equipped with this type of release film is that, in particular when the release film equipped with an adhesive is brought together and bonded with the ingoing product, the preferably luminescent registration marks applied outside of or on the edge region of the siliconized areas of the release films can cause errors in the positioning of the adhesive release films and of the ingoing products in relation to one another, and can thus cause a not inconsiderable amount of scrap. Additional production steps are moreover required, since the registration marks usually have to be applied by printing onto the silicone coating of the release films, with a resultant increase in the production time for the release films. This type of print moreover requires the use of printing blocks and corresponding consumption of printing ink, and therefore increased production costs.

There is therefore a need for release films which permit easier production of products equipped with this type of release film, e.g. appropriate sanitary or hygiene products, which can therefore avoid the abovementioned disadvantages.

It was therefore an object of the present invention to provide a release film or protective film which permits production, with precise registration, of these types of products equipped with release films or protective films, e.g. sanitary or hygiene products, while the release film or protective film does not have luminescent (registration) marks preferably applied at prescribed intervals outside of the regions provided with a silicone coating on the release film or protective film, or applied on the silicone coating.

### SUMMARY OF THE INVENTION

Said object is achieved by providing a release film or protective film, whose silicone coating is respectively composed, at least at regular intervals, of a luminescent silicone coating section, preferably only of luminescent silicone coating sections arranged at regular intervals. A preferred feature of the release film or protective film of the invention is that it has no luminescent marks, in particular no luminescent key (registration), control, and/or print marks, other than the luminescent silicone coating sections.

With the aid of a release film or protective film thus equipped it is possible, even without registration marks, to use precise registration control to achieve not only exact positioning of the release film or protective film in relation to the ingoing products, preferably ingoing sanitary or hygiene products, but also the further processing of these products bonded with the release film or protective film. The use of the release film or protective film of the invention moreover markedly reduces the scrap rate during the production of correspondingly equipped products.

### DETAILED DESCRIPTION

For the purposes of the present invention, the term “luminescent” describes at least those sections of the silicone coating of the release film or protective film of the invention that after excitation by energy input—e.g. by absorption of UV radiation—can emit electromagnetic radiation, produced during transition from an electronically excited state to a lower-energy state, e.g. the unexcited state, where the electromagnetic radiation emitted is generally of lower energy, i.e. of longer wavelength, than the electromagnetic radiation previously absorbed. The electromagnetic radiation emitted here can have a wavelength within the wavelength range of UV radiation, preferably in a wavelength range from 200 to 380 nm, in the wavelength range of visible light, preferably in a wavelength range from 380 to 780 nm, particularly preferably in a wavelength range from 400 to 500 nm, and/or in the wavelength range of infrared radiation, preferably in a wavelength range >780 nm, particularly preferably from 780 nm to 1 mm. A distinction may be made between two types of luminescence: namely fluorescence and phosphorescence. These terms are known to the person skilled in the art.

It is preferable that each section of the silicone coating of the release film or protective film of the invention is a fluorescent silicone coating section.

It is preferable that, in order to achieve the luminescence properties, each of the luminescent sections of the silicone coating of the release film or protective film of the invention comprises at least one luminescent compound, preferably at least one fluorescent compound, particularly preferably at least one fluorescent optical brightener.

The compounds used as luminescent compounds in the invention are preferably inorganic or organic compounds that are particularly preferably selected from the group consisting of inorganic dyes, inorganic pigments, organic dyes, and organic pigments, very particularly preferably selected from the group consisting of organic dyes and organic pigments which respectively can luminesce, preferably fluoresce or phosphoresce, particularly preferably fluoresce.

A preferably suitable luminescent compound used in the invention is at least one compound which luminesces on excitation with UV radiation, preferably on excitation with UV radiation in a wavelength range from 200 to 400 nm.

A preferably suitable luminescent compound used in the invention is at least one compound which, on excitation with UV radiation, emits light in the wavelength range of visible and/or infrared electromagnetic radiation, particularly preferably emits light in the wavelength range of visible electromagnetic radiation, particularly preferably in a wavelength range from 400 to 500 nm.

For the purposes of the present invention, the term “optical brightener” designates a fluorescent compound or a mixture of different fluorescent compounds which absorbs electromagnetic radiation in the wavelength range of UV radiation and has a fluorescence emission in the range of visible light, preferably in a wavelength range from 400 to 500 nm, particularly preferably in a wavelength range from 400 to 480 nm.

The luminescent compound is preferably one selected from the group consisting of stilbene compounds, ethylene compounds, coumarin compounds, naphthalimide compounds, pyrazoline compounds, pyrazole compounds, and condensed and/or conjugated aryl and/or heteroaryl compounds, where each of said compounds can optionally have substitution. Some compounds of this type are marketed, e.g. Tinopal® OB from Ciba or UAA 00105 from Xsys.

The luminescent sections of the silicone coating of the release film or protective film of the invention have been equipped with at least one luminescent compound.

In a preferred embodiment, one of the surface layers of the release film or protective film of the invention has a silicone coating which is composed, at least at regular intervals, preferably at regular intervals, of a luminescent coating section, i.e. is a silicone coating sectionally composed, at least at regular intervals, preferably at regular intervals, of a luminescent silicone coating.

In another preferred embodiment, the release film or protective film of the invention has, as one of its surface layers, a luminescent silicone coating composed only of luminescent coating sections arranged at regular intervals.

The design of each of the luminescent coating sections can be such as to cover an entire area or can correspond only to the form of the final product.

In one preferred embodiment, the luminescent sections of the silicone coating of the release film or protective film of the invention comprise from 0.1 to 15% by weight, particularly preferably from 0.5 to 10% by weight, based on the total weight of the silicone coating, of at least one luminescent compound.

In another preferred embodiment, the silicone coating of the release film or protective film of the invention comprises from 0.1 to 15% by weight, particularly preferably from 0.5 to 10% by weight, based on the total weight of the silicone coating, of at least one luminescent compound.

It is preferable that the silicone coating of the release film or protective film of the invention is a silicone coating based on at least one hardened polysiloxane.

For the purposes of the present invention, the term “polysiloxane” describes compounds whose polymer chains are composed of alternating silicon atoms and oxygen atoms. A polysiloxane is based on n repeating siloxane units (—[Si (R₂)—O]—)ₙ, each of which mutually independently has disubstitution with two organic moieties R, where each R is preferably R₁ or OR¹, and each R¹ is an alkyl moiety or an aryl moiety. It is preferable that the hardened polysiloxane of the invention is based on a repeating dialkylsiloxane unit or on a repeating alkylarylsiloxane unit. As a function of the number of Si—O bonds present in an individual siloxane unit, in each case based on a tetravalent silicon atom, these units can be classified as terminal monofunctional siloxanes (M) having one Si—O bond, difunctional siloxanes (D) having two Si—O bonds, trifunctional siloxanes (T) having three Si—O bonds, and tetrafunctional siloxanes (Q) having four Si—O bonds. It is preferable that the polysiloxane used in the invention has a crosslinked ring or crosslinked chain structure, particularly preferably a crosslinked chain structure, linked via (D), (I), and/or (Q) units to give a two- or three-dimensional network. The number n of repeating siloxane units (—[Si(R₂)—O]—)ₙ in the polysiloxane chain is termed the degree of polymerization of the polysiloxane.

The silicone coating of the release film or protective film of the invention is preferably based on at least one hardened, i.e. crosslinked, polysiloxane selected from the group consisting of addition-crosslinked, preferably metal-catalyzed-addition-crosslinked, condensation-crosslinked, free-radical-crosslinked, cationically crosslinked, and/or moisture-crosslinked polysiloxanes.

It is preferable that the silicone coating of the release film or protective film of the invention is based on at least one hardened polysiloxane hardened via thermal hardening, via hardening with electromagnetic radiation, preferably UV radiation, or via exposure to moisture.

It is preferable that the silicone coating of the release film or protective film of the invention is based on at least one hardened polysiloxane selected from the group consisting of polydialkylsiloxanes, preferably polydimethylsiloxanes, and polyalkylaryl-siloxanes, preferably polymethylphenylsiloxanes, in each case hardened.

Thermally hardened polysiloxanes are obtained via thermal hydrosilylation of polysiloxanes having silane functions, with a compound having at least one carbon double bond. In the case of polysiloxanes hardened via electromagnetic radiation, the crosslinking of the polysiloxanes has been achieved via electromagnetic radiation, preferably via UV radiation. The polysiloxanes crosslinked via exposure to moisture, preferably to water, are obtained via a polycondensation reaction in which at least one silane function and at least one alkoxy group or at least one alkoxysilane group form an Si—O bond, with elimination of at least one molecule of alcohol. The polysiloxanes to be hardened therefore always have the functional groups that react with one another and that are required for crosslinking.

The layer thickness of the silicone coating of the release film or protective film of the invention is preferably ≦5 μm, particularly preferably ≦2 μm, very particularly preferably in the range from 0.1 μm to 1.5 μm.

It is preferable that the release film or protective film of the invention has a single-side silicone coating, i.e. has this only as one of its surface layers. As an alternative, the release film or protective film of the invention can have a double-side silicone coating, where only one silicone coating has the luminescent silicone coating sections.

The release film or protective film of the invention can optionally have one or more layers, preferably at least one backing layer and/or at least one barrier layer, preferably an oxygen-barrier layer, water-vapor-barrier layer, or oil-barrier layer.

In one preferred embodiment, the release film or protective film of the invention has at least one layer, preferably at least one layer based on at least one thermoplastic polymer, particularly preferably at least one backing layer based on at least one thermoplastic polymer and having respectively, at least at regular intervals, a luminescent silicone coating section.

A material preferably suitable for producing the substrate layer(s) of the release film or protective film of the invention is at least one thermoplastic polymer, particularly at least one thermoplastic polymer selected from the group consisting of polyolefins, polyamides, polyesters, and copolymers of at least two monomers of the polymers mentioned.

Materials that can preferably be used for producing the substrate layer(s) are thermoplastic olefin homo- or copolymers of α,β-unsaturated olefins having from 2 to 10 carbon atoms, e.g. polyethylenes (PE, in particular LDPE or HDPE), polypropylenes (PP), polybutylenes (PB), polyisobutylenes (PI), or mixtures of at least two of the polymers mentioned. “LDPE” designates low-density polyethylene, with density in the range from 0.86 to 0.93 g/cm³ and featuring a high degree of branching of the molecules. “HDPE” designates high-density polyethylene having only a small amount of branching of the molecular chain, where the density can be in the range from 0.94 to 0.97 g/cm³. Preferred polyolefins for producing the substrate layer(s) are ethylene homo- or copolymers and propylene homo- or copolymers.

If polyamides are used for producing the substrate layer(s), suitable materials are thermoplastic aliphatic, partially aromatic or aromatic polyamide homo- or copolymers. Polyamides of this type are polyamides derived from diamines, for example aliphatic diamines having from 2 to 10 carbon atoms, in particular hexamethylenediamine, or from aromatic diamines having 6 to 10 carbon atoms, in particular p-phenylenediamine, and from dicarboxylic acids, for example aliphatic or aromatic dicarboxylic acids having from 6 to 14 carbon atoms, e.g. adipic acid, terephthalic acid or isoterephthalic acid. The polyamides can moreover have been produced from lactams having from 4 to 10 carbon atoms, e.g. ε-caprolactam. Examples of polyamides particularly suitable are PA 6, PA 12, PA 66, PA 6I, PA 6T and/or mixtures of at least two of the polyamides mentioned.

Polyesters that can be used for producing the substrate layer(s) are thermoplastic, aliphatic, partially aromatic or aromatic ester homo- or copolymers. Polyesters of this type derive from polyols, e.g. ethylene glycol or 1,4-butanediol, and from dicarboxylic acids or dicarboxylic acid derivatives, for example adipic acid, and/or terephthalic acid. It is also possible according to the invention to use polycarbonates (PC) as polyesters for producing the substrate layer(s). Materials preferably suitable are polybutylene adipate (PBA), polyethylene terephthalate (PET), polybutylene terephthalate (PBT), or the corresponding copolymers.

A material preferably suitable for producing a barrier layer(s) of the release films or protective films of the invention is at least one thermoplastic polymer, particularly at least one thermoplastic polymer selected from the group consisting of polyamides, ethylene-vinyl alcohol copolymers (EVOH), polyvinyl alcohols (PVOH), and a mixture of at least two of the polymers mentioned.

The polyamides (PA) used for producing the barrier layer(s) are preferably the same as those that can be used for producing the substrate layer(s) of the release film or protective film of the invention.

The polyvinyl alcohols used for producing the barrier layer(s) are obtained via complete or incomplete hydrolysis of corresponding polyvinyl acetates (PVA) and therefore comprise not only partially hydrolyzed polyvinyl acetates with a degree of hydrolysis of from 50 to 98 mol % but also completely hydrolyzed polyvinyl acetates with a degree of hydrolysis 98%.

The ethylene-vinyl alcohol copolymers (EVOH) used for producing the barrier layer(s) are obtained via complete or incomplete hydrolysis of corresponding ethylene-containing polyvinyl acetates (EVAc) and comprise especially completely hydrolyzed ethylene-containing polyvinyl acetates with a degree of hydrolysis 98%.

The silicone coating, and also optionally the substrate layer(s), and/or barrier layer(s) of the release film or protective film of the invention can, if necessary, respectively mutually independently have additions of additives selected from the group consisting of antiblocking agents, antistatic agents, antifogging agents, antimicrobial ingredients, dyes, color pigments, stabilizers, preferably heat stabilizers and process stabilizers, processing aids, flame retardants, nucleating agents, crystallization agents, preferably crystal-nucleating agents, lubricants, optical brighteners, flexibilizing agents, sealing agents, plasticizers, spacers, fillers, peel additives, waxes, wetting agents, surface-active compounds, preferably surfactants, and dispersing agents. It is essential here to retain the release effect of the silicone coating and the luminescent property thereof.

The silicone coating, and also optionally the substrate layer(s), and/or barrier layer(s) of the release film or protective film of the invention can, if necessary, respectively mutually independently comprise at least 0.01-30% by weight, preferably at least 0.1-20% by weight, based in each case on the total weight of an individual layer, of at least one of the abovementioned additives.

The present invention also provides a process for producing the release film or protective film of the invention.

The release film or protective film of the invention can be produced by known production processes, e.g. lamination or (co)extrusion, preferably by coextrusion. Substrate layer(s) and/or optionally present barrier layer(s) of the release film or protective film of the invention can be formed by extrusion, individually preferably by flat-film extrusion (cast extrusion), or blown-film extrusion, in particular by flat-film coextrusion (cast coextrusion), or blown-film coextrusion.

In a preferred embodiment, the substrate layer(s) and/or optionally present barrier layer(s) of the release film or protective film of the invention can be produced and processed as optionally multilayer film composite in the form of a tubular film. The resultant whole optionally multilayer film composite in the form of a tubular film can be embossed directly thereafter without prior storage, or, as an alternative, during production of said film. It is preferable that the optionally multilayer film composite in the form of a tubular film is embossed during production of said film.

In another preferred embodiment, the substrate layer(s) and/or optionally present barrier layer(s) of the release film or protective film of the invention can be produced and processed as optionally multilayer cast film composite. The resultant whole optionally multilayer film composite in the form of a tubular film can be embossed directly thereafter without prior storage, or, as an alternative, during production of said film. It is preferable that the optionally multilayer film composite in the form of a tubular film is embossed during production of said film.

It is preferable that at least one layer, particularly at least one substrate layer, or one layer composite made of at least one substrate layer and/or of at least one barrier layer, is coated on a surface (in the case of the layer composite preferably on the surface of a substrate layer of the layer composite) with a silicone coating composed, at least at regular intervals, of a luminescent section. Therefore the surface of at least one layer or of the before layer composite is equipped, at least in regular coating sections, preferably only at regular intervals, with a preferably liquid mixture of at least one not yet hardened polysiloxane and of at least one luminescent compound, and optionally of at least one beforementioned additive. This is achieved via coating the surface of said layer or of said layer composite with the mixture at regular intervals or optionally in alternation with a luminescence-free coating composition across the entire surface of the continuous film web, preferably at regular intervals. Therefore the abovementioned preferably liquid mixture here can be applied at regular intervals over an entire area, i.e. over the entire film width, or only in accordance with a printing block, on the film web. The design or form of the luminescent sections of the silicone coating here can preferably be adapted to be appropriate to the form of the ingoing product to be equipped with the release film or protective film, and the printing block can preferably be changed accordingly as required. It is therefore preferable that all of the luminescent sections of the silicone coating in a production batch have the same design or form. The hardening of the mixture made of the yet not hardened polysiloxane and the luminescent compound or of the mixture made of the yet not hardened polysiloxane, of the luminescent compound and optionally of an abovementioned additive, and optionally of a luminescence-free silicone coating composition is preferably achieved via exposure to heat or with the aid of electromagnetic radiation, preferably UV radiation, or via moisture, optionally via addition of at least one UV initiator and/or one free-radical initiator to the unhardened polysiloxane or into the mixture, particularly preferably via exposure to heat or to electromagnetic radiation, preferably UV radiation. The release film or protective film of the invention, thus equipped, can optionally then be cut to the desired dimensions.

The release film or protective film of the invention is preferably an embossed film.

The release film or protective film of the invention can have a non-luminescent print and/or can be colored and/or transparent.

A feature of the release film or protective film of the invention is excellent haptic properties and improved optical properties in comparison to the known release and protective films.

The release film or protective film of the invention is suitable for use on preferably sheet-like ingoing products, preferably appropriate ingoing sanitary or hygiene products. To this end, it is preferably that all of the luminescent sections of the silicone coating of the release film or protective film of the invention have the same design or form. It is preferably that said design or form has been adapted to be appropriate to the design or form of the ingoing products to be equipped with a release film or protective film of the invention, preferably sheet-like ingoing products, preferably appropriate ingoing sanitary or hygiene products.

The present invention also provides a preferably continuous process for equipping preferably sheet-like ingoing products, preferably appropriate ingoing sanitary or hygiene products, with a release film or protective film of the invention.

The process of the invention for equipping preferably sheet-like ingoing products, preferably appropriate ingoing sanitary or hygiene products, with a release film or protective film of the invention comprises at least the following steps:
(a) synchronized, separate introduction of discrete ingoing products, preferably ingoing sanitary or hygiene products, and of a release film or protective film whose silicone coating is respectively composed, at least at regular intervals, of a luminescent silicone coating section, optionally respectively in alternation with a non-luminescent silicone coating section, preferably composed only of luminescent silicone coating sections arranged at regular intervals and preferably corresponding in their design and form to the product to be equipped therewith,
(b) at least partially equipping each of said luminescent sections of the silicone coating of the release film or protective film with a preferably pressure-sensitive adhesive, preferably in the form of at least one strip, or at least partially equipping each of the discrete ingoing products, preferably ingoing sanitary or hygiene products, with a preferably pressure-sensitive adhesive, preferably in the form of at least one strip,
(c) bringing together and connecting each of the luminescent sections of the silicone coating being at least partially equipped with a preferably pressure-sensitive adhesive with a discrete ingoing product, preferably an ingoing sanitary or hygiene product, or bringing together and connecting each of the discrete ingoing products being at least partially equipped with a preferably pressure-sensitive adhesive, preferably ingoing sanitary or hygiene product, with a luminescent section of the silicone coating of the release film or protective film,
(d) optionally onward conveying of the products bonded with the release film or protective film, preferably sanitary or hygiene products,
(e) optionally folding, optionally sealing and separating the products equipped with the release film or protective film, preferably the sanitary or hygiene products,
(f) optionally packaging each product equipped with a release film or protective film, preferably sanitary or hygiene product, and optionally further separating the packaged products, preferably sanitary or hygiene products,
   where at least the steps (b) and (c) and optionally also the step (a) are monitored precisely by at least one sensor element detecting luminescence, preferably fluorescence.

Suitable adhesives used in the step (b) of the process of the invention are preferably pressure-sensitive adhesives, particularly preferably conventional pressure-sensitive hot-melt adhesives known to the person skilled in the art, e.g. pressure-sensitive hot-melt adhesives based on thermoplastic rubber. It is particularly preferable to use pressure-sensitive hot-melt adhesives in the step (b) of the process of the invention.

It is preferably that, in the step (b), each of the luminescent sections of the silicone coating of the release film or protective film of the invention, or each of the ingoing products, preferably ingoing sanitary or hygiene products, is equipped at least partially with at least one preferably pressure-sensitive adhesive, preferably in the form of at least one strip.

In a preferred embodiment of the process of the invention, in the step (b), each of the luminescent sections of the silicone coating of the release film or protective film is at least partially equipped with a preferably pressure-sensitive adhesive and, in the step (c), each luminescent section that is at least partially equipped with a preferably pressure-sensitive adhesive, which means each adhesive, luminescent section of the silicone coating of the release film or protective film is brought together and connected with a discrete ingoing product, preferably an ingoing sanitary or hygiene product.

In another preferred embodiment of the process of the invention, in the step (b), each of the discrete ingoing products, preferably ingoing sanitary or hygiene products, is at least partially equipped with a preferably pressure-sensitive adhesive and, in the step (c), each discrete ingoing product that has at least partially been equipped with a preferably pressure-sensitive adhesive, which means each adhesive, preferably ingoing sanitary or hygiene product, is brought together and connected with a luminescent section of the silicone coating of a release film or protective film of the invention.

In the step (e), the products, preferably sanitary or hygiene products, equipped with the release film or protective film of the invention and optionally packaged therein, are optionally folded and separated optionally by sealing. The separation can be achieved via suitable perforation, cutting and/or punching (from the material or through the material) of the products, preferably sanitary or hygiene products, equipped with the release film or protective film of the invention. If the separation is achieved via punching, the products, preferably sanitary or hygiene products, here equipped with the release film or protective film of the invention are preferably passed continuously into a punching system and positioned under the punching head of the punching machine. With the aid of the punch of the punching machine, the products, preferably sanitary or hygiene products, equipped with the release film or protective film of the invention, are perforated or punched out at an adjustable rate (punching rate).

An element preferably suitable as at least one sensor element that detects luminescence, preferably fluorescence, and that monitors at least the steps (b) and (c) and optionally also the step (a), preferably the steps (a) to (c), precisely can be any conventional UV-radiation-based sensor element that detects luminescence, preferably fluorescence, particularly preferably any conventional luminescence detector, very particularly preferably any conventional fluorescence detector. An example of this type of luminescence detector is the LUT3-820 luminescence detector from the LUT3-8 product line from the company SICK.

The present invention further provides a preferably sheet-like product, preferably a sanitary or hygiene product, which has been equipped with a release film or protective film of the invention, and which is obtainable by a process of the invention.

It is preferable that the sanitary or hygiene product of the invention is one selected from the group consisting of sanitary napkins and pantyliners.

The present invention further provides an apparatus for carrying out the process of the invention.

The apparatus of the invention for carrying out the process of the invention here comprises at least
(1) a first conveying means for conveying the discrete ingoing products, preferably ingoing sanitary or hygiene products,
(2) a second conveying means for conveying a release film or protective film of the invention, which silicone coating is composed, at least at regular intervals, of a luminescent section, and optionally in alternation of a non-luminescent silicone coating section, preferably being composed only of luminescent silicone coating sections arranged at regular intervals,
(3) a dosing equipment for equipping each of the luminescent sections of the silicone coating of the release film or protective film at least partially with a preferably pressure-sensitive adhesive, or for equipping each discrete ingoing product, preferably ingoing sanitary or hygiene product, at least partially with a preferably pressure-sensitive adhesive,
(4) a deflector equipment for bringing the first conveying means together with the second conveying means and for connecting each of the luminescent section of the silicone coating being at least partially equipped with a preferably pressure-sensitive adhesive with each of the discrete ingoing products, preferably ingoing sanitary or hygiene product, or for connecting each of the discrete ingoing products, preferably ingoing sanitary or hygiene products, at least partially equipped with a preferably pressure-sensitive adhesive, with a luminescent section of the silicone coating of a release film or protective film of the invention,
(5) optionally a folding unit for folding each product, preferably each sanitary or hygiene product, and
(6) optionally a sealing apparatus and a punching apparatus and/or cutting apparatus for separating the products, preferably sanitary or hygiene products, and
(7) optionally a packaging unit for packaging each product, preferably each sanitary or hygiene product,
(8) and a sensor element sensitive to luminescence, preferably fluorescence, in direct or indirect contact with
(9) a control unit for monitoring precisely at least the two conveying means, the dosing equipment, the deflection equipment, the punching and/or cutting apparatus, and optionally the packaging unit.

In one preferred embodiment of the apparatus of the invention, the dosing equipment (3) is dosing equipment for equipping, at least partially, each of the luminescent sections of the silicone coating of the release film or protective film with a preferably pressure-sensitive adhesive, and the deflector equipment (4) is a deflector equipment for bringing the first conveying means together with the second conveying means and for connecting each of the luminescent sections of the silicone coating being at least partially equipped with a preferably pressure-sensitive adhesive with a discrete ingoing product, preferably an ingoing sanitary or hygiene product.

In another preferred embodiment of the apparatus of the invention, the dosing equipment (3) is dosing equipment for equipping, at least partially, each discrete ingoing product, preferably ingoing sanitary or hygiene product, with a preferably pressure-sensitive adhesive, and the deflector equipment (4) is a deflector for bringing the first conveying means together with the second conveying means and for connecting each of the discrete ingoing products, preferably ingoing sanitary or hygiene products, being at least partially equipped with a preferably pressure-sensitive adhesive, with a luminescent section of the silicone coating, preferably whereby the design and form of a release film or protective film of the invention corresponds to the design and form of the ingoing product to be used.

What is claimed is:

1. A release film or protective film having a silicone coating, which silicone coating is composed, at least at regular intervals, of a luminescent silicone coating section comprising at least one luminescent compound, wherein the luminescent silicone coating sections comprise from 0.1 to 15% by weight, based on the total weight of the silicone coating, of the luminescent compound.

2. The release film or protective film as claimed in claim 1, wherein the silicone coating is composed only of the luminescent silicone coating sections comprising at least one luminescent compound and arranged at regular intervals.

3. The release film or protective film as claimed in claim 1, wherein each of the luminescent sections comprising at least one luminescent compound in the silicone coating is a fluorescent silicone section.

4. The release film or protective film as claimed in claim 1, wherein each of the luminescent silicone sections of the silicone coating comprises at least one fluorescent compound as luminescent compound.

5. The release film or protective film as claimed in claim 1, wherein one of the surfaces of the release film or protective film has a silicone coating which is composed, at least at regular intervals, of a luminescent silicone coating section comprising at least one luminescent compound, and optionally, respectively in alternation, of a non-luminescent silicone coating section, or is composed only of luminescent coating sections comprising at least one luminescent compound and arranged at regular intervals.

6. The release film or protective film as claimed in claim 1, wherein the luminescent sections of the silicone coating comprise from 0.5 to 10% by weight, based on the total weight of the silicone coating, of the luminescent compound.

7. The release film or protective film as claimed in claim 1, wherein the release film or protective film comprises no luminescent markings other than the luminescent silicone coating sections comprising at least one luminescent compound.

8. The release film of protective film as claimed in claim 1, wherein the design or form of the luminescent silicone coating sections corresponds to that of the product to be equipped with the release film or protective film.

9. A process for equipping a sheet-like ingoing product with a release film or protective film as claimed in claim 1, comprising the steps of:
(a) synchronized, separate introduction of discrete ingoing products, and of the release film or protective film,
(b) at least partially equipping each of said luminescent silicone coating sections of the release film or protective film comprising at least one luminescent compound with a pressure-sensitive adhesive, optionally in the form of at least one strip, or at least partially equipping each of the discrete ingoing products with a pressure-sensitive adhesive, optionally in the form of at least one strip;
(c) bringing together and connecting each luminescent silicone coating section being at least partially equipped with a pressure-sensitive adhesive and comprising at least one luminescent compound with an ingoing product, or bringing together and connecting each discrete ingoing product, being at least partially equipped with a pressure-sensitive adhesive, with respectively a luminescent silicone coating section comprising at least one luminescent compound of the release film or protective film,
(d) optionally onward conveying each product connected with the release film or protective film,
(e) optionally folding and optional sealing and separating each product equipped with the release film or protective film,
(f) optionally packaging of each product equipped with a release film or protective film, and optional further separating of the packaged products,
where at least the steps (b) and (c) and optionally also the step (a) are monitored precisely by at least one sensor element detecting luminescence.

10. The process of claim 9 wherein said sheet-like ingoing product is an ingoing sanitary or hygiene product.

11. The process as claimed in claim 9, wherein the process is continuous.

12. A sheet-like sanitary or hygiene product, obtained by the process of claim 9.

13. The sheet-like sanitary or hygiene product of claim 12, wherein the sanitary or hygiene product is a sanitary napkin or a pantyliner.

14. An apparatus for carrying out a process as claimed in claim 9 comprising:
(1) a first conveying means for conveying the discrete ingoing products,
(2) a second conveying means for conveying a release film or protective film having a silicone coating composed, at least at regular intervals, of a luminescent silicone coating section comprising at least one luminescent compound, where the design or form of the luminescent coating sections corresponds to that of the product to be equipped with the release film or protective film, and the luminescent silicone coating sections comprise from 0.1 to 15% by weight, based on the total weight of the silicone coating, of the luminescent compound,
(3) a dosing equipment for equipping, at least partially, each of the luminescent silicone coating sections with a pressure-sensitive adhesive, or for equipping, at least partially, each discrete ingoing product, optionally discrete sanitary or hygiene product, with a pressure-sensitive adhesive,
(4) a deflector equipment for bringing the first conveying means together with the second conveying means and for connecting each luminescent silicone coating section being at least partially equipped with a pressure-sensitive adhesive and comprising at least one luminescent compound with a discrete ingoing product, optionally ingoing sanitary or hygiene product, or for connecting each of the discrete ingoing products, optionally ingoing sanitary or hygiene products, being at least partially equipped with a pressure-sensitive adhesive, with a luminescent silicone coating section comprising at least one luminescent compound,
(5) optionally a folding unit for folding each of the products obtained according to (4),
(6) optionally a sealing apparatus and a punching apparatus, and/or cutting apparatus for separating the products obtained according to (4) or (5), and
(7) optionally a packaging unit for packaging each of the products obtained according to (4), (5) or (6),
(8) and a sensor element sensitive to luminescence, in direct or indirect contact with
(9) a control unit for monitoring precisely at least the two conveying means, the dosing equipment, the deflection equipment, the punching and/or cutting apparatus, and optionally the packaging unit.
